(19) United States
(12) Patent Application Publication
(10) Pub. No.: US 2006/0134132 A1
(43) Pub. Date: Jun. 22, 2006

(54) Protective barriers for micronutrients, phytochemicals, and nutraceuticals

(76) Inventors: Jackie McDonald Watkins, San Diego, CA (US); Michael Arthur Takaki, San Diego, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/015,651

(22) Filed: Dec. 20, 2004

(51) Int. Cl. A61K 36/185; A61K 47/00

(52) U.S. Cl. 424/195.18; 424/439

(57) ABSTRACT

Compositions that act as a protective barrier for micronutrients, phytochemicals, and nutraceuticals such as may be found in food products, for example food additives. Also, a process for producing food additives using the compositions.

### FIELD OF THE INVENTION

Embodiments of the present invention relate generally to methods of stabilizing food additives and food articles. In particular, embodiments of the present invention relate to compositions and methods for stabilizing micronutrients, phytochemicals, nutraceuticals, and other beneficial compounds in food additives and food articles.

### BACKGROUND OF THE INVENTION

Society has become increasingly aware of the importance of maintaining proper nutritional habits. As the average diet increasingly utilizes processed foods, it has become more important for processed foods to provide the same good nutritional benefits of natural foods.

Nutrients necessary to support life include proteins, carbohydrates, fats, minerals, and vitamins. Processed food products are often supplemented by the addition of synthetic nutrients to help replace natural nutrients that may have been rendered inactive or otherwise damaged during processing of the food product. Elevated temperatures during cooking, for example, may damage the natural nutrients that are present in foods. The freeze drying process by which food products are dehydrated also may damage the natural nutrients that are present in foods.

Besides nutrients, other non-nutritive compounds found in, for example, fruits and vegetables may have beneficial effects when consumed. Nutraceuticals, for example, are chemical compounds in foods that may aid in preventing or treating diseases and other medical conditions when consumed even though they are not traditionally recognized to posses nutritive value.

Phytochemicals are chemical compounds in plants that also may aid in preventing or treating diseases and other medical conditions when consumed even though they also are not traditionally recognized to posses nutritive value. Nutraceuticals and phytochemicals, like nutrients, may be damaged by subsequent processing of food products to which they are added.

U.S. application Ser. No. 10/391,747, the disclosure of which is incorporated by reference herein in its entirety, discloses a process for stabilizing and preserving nutrients, particularly phytochemicals. The process involves application of a colloid plant extract selected from vegetable gums, hydroscopic phosphatides, vegetable albumin, and pectin. This process is not entirely satisfactory from the standpoint of maintaining the nutritional value of the original foodstuffs, since a relatively large percentage of the nutritional value still may be lost during subsequent processing.

The description herein of problems and disadvantages of known apparatus, methods, and compositions is not intended to limit the invention to the exclusion of these known entities. Indeed, embodiments of the invention may include one or more of the known apparatus, methods, and compositions without suffering from the disadvantages and problems noted herein.

### SUMMARY OF THE INVENTION

What is needed is a composition that stabilizes and protects micronutrients, phytochemicals, nutraceuticals, and other beneficial compounds in food additives and food articles from degradation during processing.

Therefore, in accordance with a feature of an embodiment of the present invention, there is provided a composition for stabilizing and protecting micronutrients, phytochemicals, nutraceuticals, and other beneficial compounds in food additives and food articles. The composition may comprise a mixture of vegetable gums, oligosaccharides, and whey protein.

In accordance with still another embodiment of the present invention there is provided a process for producing a food additive. A food article may be chopped; disinfected; sprayed with a mixture of vegetable gums, oligosaccharides, and whey protein; and then freeze dried to provide a stabilized freeze dried food article suitable for subsequent processing into a food additive, for example by grinding into a powder.

Still further features and advantages of the present invention are identified in the ensuing description.

### DETAILED DESCRIPTION OF THE INVENTION

The following description is intended to convey a thorough understanding of embodiments of the present invention by providing a number of specific embodiments and details involving protective barriers for micronutrients, phytochemicals, and nutraceuticals. It is understood, however, that the various embodiments of the present invention are not limited to these specific embodiments and details, which are exemplary only. It is further understood that one possessing ordinary skill in the art, in light of known systems and methods, would appreciate the use of the invention for its intended purposes and benefits in any number of alternative embodiments.

As used throughout this description, “nutrients” refers to compounds generally recognized as necessary to support human life. These compounds include proteins, carbohydrates, fats, minerals, and vitamins. Mineral and vitamins, because they are generally required in much smaller amounts than the other nutrients, may be referred to as “micronutrients.”

“Nutraceuticals,” as used herein, refers to non-nutritive compounds that nonetheless may produce beneficial effects, for example medicinal effects, when consumed. Exemplary nutraceuticals include, but are not limited to, phytochemicals, glucosamine, methylsulfonylmethane, chondroitin, ruscus, bromlein, boswellin, carnitine, hydroxycitric acid, chitosan, acetyl-L-carnitine, phosphatidylserine, huperzine-A, S-adenosylmethione, vinceptine, DMAE, lecithins, ginseng, ashwagandha, ipriflavone, NADH, magnesium malate, and D-ribose. “Nutraceuticals” also include as yet unknown or unidentified compounds that may produce beneficial effects when consumed.

“Phytochemicals,” as used herein, refers to non-nutritive plant chemicals that nonetheless may produce beneficial effects when consumed. For example, some phytochemicals have been implicated as anti-cancer compounds or may posses other medicinal qualities. Exemplary phytochemicals include, but are not limited to, ajoene, allyl sulfides, beta-carotene, butyl phthalide, calcium pectate, capsaicin, carotenoids, catechin hydrate, coumarin, coumesterol, ellagic acid, flavonoids and isoflavones such as quercetin, genistein, gingerols, glycyrrhizin catechins, heliotropin, indoles and glucosinolates, isothiocyanates and thiocyanates, kaempferol, lutein, lycopene, monoterpenes such as limonene, para-coumaric acid, phenols, phthalides, phytic acid, polyacetylenes, quercetin, saponin, silymarin, sulfaforaphane, thiols, and zeaxanthin. “Phytochemicals” also include as yet unknown or unidentified plant chemicals that may produce beneficial effects when consumed.

In an embodiment of the invention, there is provided a composition for stabilizing and protecting micronutrients, phytochemicals, nutraceuticals, and other beneficial compounds in food additives and food articles. The composition may comprise vegetable gums, oligosaccharides, and whey protein. An exemplary use of the composition is to protect micronutrients, phytochemicals, nutraceuticals, and other beneficial compounds in food additives and articles from degradation when subjected to low or high temperatures. Without intending on being bound by any theory of operation, the inventor believes that the whey proteins act as a stabilizer to protect the beneficial compounds in the food additives and articles, the oligosaccharides act as an adhesive to bind the whey proteins to the beneficial compounds of the food additives and articles, and the vegetable gum also acts as a protective barrier and biopolymer to help prevent degradation of the beneficial compounds.

Any applicable vegetable gum may be used in the composition, following the guidelines provided herein. Vegetable gums contemplated for use in the invention include, but are not limited to, the following: gum arabic (acacia gum), guar gum (guar flour), agar (agar-agar), carrageenan gum (alpha, kappa and all other types), karaya gum (sterculia gum, India tragacanth, kadaya gum), gum ghatti, locust agar, algin, pectin, xanthan gum, locust bean gum, gum tragacanth, tamarind gum, and combinations and mixtures thereof. Additionally, modified vegetable gums may be used in accordance with the present invention. Modified vegetable gums contemplated for use in the invention include, but are not limited to, the following: chelated agar; pectin derivatives including both low- and high-methoxyl pectin; alginates such as propylene glycol alginate; cellulose derivatives such as microcrystalline cellulose, methylcellulose, sodium carboxymethyl cellulose, carboxymethylcellulose, hydroxypropyl cellulose, hydroxypropyl methyl cellulose, and sodium hydroxymethyl cellulose; carboxymethyl locust bean gum; gellan gum; carboxymethyl guar gum; and combinations and mixtures thereof.

In a preferred embodiment, the vegetable gum is gellan gum. Gellan gum is a polysaccharide of repeating tetrasaccharide units. Each tetrasaccharide unit has two glucose residues, one glucuronic acid, and one rhamnose residue. Additionally, the tetrasaccharide units may be substituted with acyl (glyceryl and acetyl) groups at the O-glycosidically-linked esters. Gellan gum is commonly obtained from fermentation of a carbohydrate by the bacteria Pseudomonas elodea, although gellan gum obtained from other sources also is applicable in the invention.

In another preferred embodiment, the vegetable gum is xanthan gum. Xanthan gum is a polysaccharide composed of glucose, mannose, and glucuronic acid and has a backbone similar to the backbone of cellulose but with additional trisaccharide sidechains. Xanthan gum is commonly used in food products to control viscosity because of its hydration and gelling capabilities. Additionally, its relatively good hydration ability at low temperatures may make xanthan gum useful in hindering ice recrystallization in freeze-thaw situations. Xanthan gum is commonly obtained from fermentation of com sugar by the bacteria Xanthomonas campestris, although gellan gum obtained from other sources also is applicable in the invention.

In yet another preferred embodiment, the vegetable gum is carrageenan gum. “Carrageenan” refers collectively to a group of polysaccharides consisting of long chains of galactose derivatives obtained by alkaline extraction from red seaweed, commonly of the genus Chondrus, Eucheuma, Gigartina and Iridaea. The three most common carrageenan gums (i.e., κ-carrageenan, I-carrageenan, λ-carrageenan) are commonly used as gels and thickeners in food products.

In preferred embodiment, the composition comprises about 1% to about 50% vegetable gums. In a more preferred embodiment, the composition comprises about 1% to about 25% vegetable gums. In a most preferred embodiment, the composition comprises about 1% to about 10% by weight vegetable gums.

Any applicable oligosaccharides may be used in the composition, following the guidelines provided herein. Oligosaccharides are short chains of sugar molecules. Common oligosaccharides include fructo-oligosaccharides (FOS), galacto-oligosaccharides (GOS), and inulins. Vegetables are common sources of oligosaccharides, though oligosaccharides obtained from other sources also are contemplated for use in the invention.

In a preferred embodiment, the composition comprises about 5% to about 45% by weight oligosaccharides. In a more preferred embodiment, the composition comprises about 5% to about 25% by weight oligosaccharides.

In a most preferred embodiment, the composition comprises about 5% to about 10% by weight oligosaccharides.

Any applicable whey proteins may be used in the composition, following the guidelines provided herein. Whey proteins are a group of proteins including beta-lactoglobulin glycomacropeptide (GMP), alpha-lactalbumin, lactoferrin, immunoglobulins, lactoperoxidase, bovine berum albumin (BSA), and lysozyme obtained from cow's milk. Whey proteins may be obtained by removing casein (i.e., the curd) from cow's milk and purifying the remaining solution to remove lactose, fat, and other constituents of cow's milk. Whey proteins obtained from alternative sources and processes also are useful in embodiments of the invention.

In a preferred embodiment, the composition comprises about 1% to about 20% by weight whey proteins. In a more preferred embodiment, the composition comprises about 5% to about 15% by weight whey proteins. In a most preferred embodiment, the composition comprises about 5% to about 10% by weight whey proteins.

Application of the composition of vegetable gums, whey proteins, and oligosaccharides to a food additive or food article may act as a protective barrier at the molecular level to increase the temperature resistant qualities of the food additive or article, particularly the temperature resistant qualities of micronutrients, phytochemicals, and nutraceuticals that may be present in the food additive or article. For example, addition of the composition may help a food additive or article to withstand the 150° F. to 180° F. temperature changes commonly associated with the freeze drying process. Food additives and articles treated with the natural composition may experience less degradation of their natural nutritional and extra-nutritional content when cooked or frozen by consumers and manufacturers. Application of the natural composition of vegetable gums, whey proteins, and oligosaccharides therefore may help to reduce reliance upon sythetic nutritional supplements in processed food products. Additionally, application of the natural composition may increase the shelf life of treated food additives and articles.

In another embodiment, there is provided a process for preparing a food additive. A food article may be shredded and sanitized. A composition comprising vegetable gums, oligosaccharides, and whey protein as described herein may be applied to the food article. The food article may be freeze dried and ground into a powder. A stabilizing composition optionally may be applied to the powder. The food additive may be mixed with processed foods to enhance the nutritional and extra-nutritional content of the food.

The food article may be any applicable raw material useful as a food additive, as will be appreciated by one skilled in the art. Raw fruits and vegetables, for example, are contemplated as food articles. Phytochemical-rich foods are preferred food articles. Phytochemical-rich foods include, but are not limited to, tomatoes, broccoli, garlic, brussel sprouts, cabbage, bok choi, and other cruciferous vegetables. Additionally, fruit such as apples and oranges are useful as food articles. The raw fruits and vegetables preferably may be selected, for example, to ensure freshness and stored at reduced temperatures. The food articles may be shredded to a particle size of about 6.2 mm (0.25 inches). Preferably, the food article may be shredded to a particle size of about 3.2 mm (0.125 inches). If desired, the shredded food articles may be selected by weight. The food article then may be sprayed with a solution of 1 ppm chlorine or another disinfectant suitable for use on a food substance. The chlorine solution may act to disinfect the food article and remove pathogenic microorganisms.

A mixture of vegetable gums, oligosaccharides, and whey protein as described herein may be applied to the food article, for example by dusting or spraying. The mixture may help to prevent degradation of nutraceuticals and phytochemicals present in the food article during subsequent processing, for example freeze drying. Preferably, the vegetable gum is selected from gellan, xanthan, carrageenan, and combinations and mixtures thereof. A mixture of about 1% to about 50% by weight vegetable gums, about 5% to about 45% by weight oligosaccharides, and about 1% to about 20% by weight whey proteins is preferred. A mixture of about 1% to about 25% by weight vegetable gums, about 5% to about 25% by weight oligosaccharides, and about 5% to about 15% by weight whey proteins is more preferred. A mixture of about 1% to about 10% by weight vegetable gums, about 5% to about 10% by weight oligosaccharides, and about 5% to about 10% by weight whey proteins is most preferred. The mixture may be applied in a liquid form or a dry powdered form. It may be preferable to apply the mixture in a temperature controlled manner so as to maximize the adhesion between the mixture and the food article. Also, it may be preferable to mix the vegetable gum and oligosaccharide components first, thereby forming a sticky product, and then add the whey protein to the sticky product and mix until homogenous.

The food article may be freeze dried to reduce the moisture content of the article. Freeze drying may proceed in any applicable manner, as will be appreciated by one skilled in the art. In a preferred embodiment, the food article may be cooled to about 0° C. (32° F.) before being introduced to a rotary type freeze dryer. After introduction of the food article, the pressure in the freeze dryer may be reduced to about 500 microns of Hg (0.5 torr), which may aid in removing moisture from the food article. The evaporation of water from the food article due to the low pressure in the freeze dryer may further reduce the temperature of the food article, for example, to about −18° C. (0° F.). The low pressure may be maintained for about 8 to about 12 hours. Thereafter, the temperature of the freeze dryer may be allowed to increase to about 30° C. (86° F.). Freeze drying the food article may preferably reduce the moisture content, which is typically about 85% before freeze drying, to within the range of about 2% to about 5%.

The food article may be ground to a powder. Preferably, the size of the powder is about 60 mesh to about 100 mesh. The mesh size of the powder may be determined by sifting the powder through a screen with appropriately sized orifices. A sifting process also may be used to separate powders of different sizes in order to obtain a powder of a desired mesh size. A stabilizing composition may optionally be added to the food article. The stabilizing composition, if used, may help to further prevent degradation of micronutrients, nutraceuticals, and phytochemicals present in the food article during subsequent processes, for example cooking. In a preferred embodiment, a stabilizing composition comprising a second mixture of vegetable gums, oligosaccharides, and whey protein as described herein is added to the food article.

The food additives made according to this process may exhibit superior resistance to degradation and deactivation of constituent nutraceuticals, phytochemicals, and micronutrients during subsequent processing of food products supplemented with the food additives. For example, the phytochemicals of the food additives made according to this process may resist temperatures up to about 80° C. (180° F.). More preferably, food additives made according to this process may maintain 97% of their natural nutritional content at temperatures up to 205° C. (400° F.).

The invention now will be described in more detail with reference to the following non-limiting example.

### EXAMPLE

Apple was diced to fragments of approximately 6.3 mm (0.25 inches) in size. The diced apple was spread into a layer about 2.2 cm (1 inch) thick and sprayed with a 1 ppm chlorine solution. The washed apple was introduced to a freezer and cooled to about 0° C. (32° F.) or until completely frozen. The cooled apple was sprayed with a mixture of liquid vegetable gums, oligosaccharides, and whey proteins and then introduced into a freeze dryer. The pressure in the freeze dryer was reduced to approximately 500 microns of Hg (0.5 torr) and maintained for about 8 to about 12 hours. The temperature of the freeze dryer during this time was about 17° C. (0° F.) and the moisture content of the apple was reduced from about 85% to about 20%. The temperature of the freeze dryer was allowed to rise to about 30° C. (86° F.). The final moisture content of the apple when removed from the freeze dryer was about 2% to about 3%.

While the description of the embodiments presented above has been described with reference to particularly preferred embodiments, it is recognized that similar advantages may be obtained by other embodiments. It will be evident to those skilled in the art that various changes and modifications can be made without departing from the spirit and scope of the present invention, and all such modifications are within the scope of this invention.

What is claimed is:

1. A composition for stabilizing and protecting micronutrients, phytochemicals, nutraceuticals, and other beneficial compounds and nutrients, comprising:
vegetable gums;
oligosaccharides; and
whey proteins.

2. The composition of claim 1, where the vegetable gum is selected from the group consisting of: gellan, xanthan, carrageenan, and combinations and mixtures thereof.

3. The composition of claim 1, where the composition comprises about 1% to about 10% by weight vegetable gums.

4. The composition of claim 1, where the composition comprises about 5% to about 10% by weight oligosaccharides.

5. The composition of claim 1, where the composition comprises about 5% to about 10% by weight whey proteins.

6. A process for stabilizing and protecting food additives or food articles comprising applying the composition of claim 1 to the food additives or food articles.

7. A process for producing a food additive comprising:
shredding a food article;
sanitizing the food article;
applying a composition of claim 1 to the food article;
freeze drying the food article; and
grinding the food article into a powder to form a food additive.

8. The process of claim 7, where the food article is a fruit or vegetable.

9. The process of claim 7, where shredding the food article comprises slicing, cutting, chopping, cubing, mincing, or dicing the food article to about 6.2 mm (0.25 inches) particle size.

10. The process of claim 7, where sanitizing the food article comprises spraying a solution of about 1 ppm chlorine on the food article.

11. The process of claim 7, where applying a composition of claim 1 to the food article comprises spraying a liquid solution or dusting a dry mixture of the composition of claim 1 on the food article.

12. The process of claim 7, where freeze drying the food article comprises:
introducing the food article at about 0° C. (32° F.) to a freeze dryer;
lowering the pressure in the freeze dryer to about 500 micron Hg (0.5 torr);
maintaining the lowered pressure for about 8 to about 12 hours; and
gradually raising the temperature to about 30° C. (86° F.).

13. The process of claim 7, where freeze drying the food article reduces the moisture content of the food article to between about 2% and about 5%.

14. The process of claim 7, where grinding the food article comprises pulverizing, crushing, or pounding the food article to form a powder of about 60 mesh to about 100 mesh size.
